(19) United States
(12) Patent Application Publication
(10) Pub. No.: US 2026/0327170 A1
(43) Pub. Date: Oct. 22, 2026

(54) DIRECT SYNTHESIS OF LIQUID FUEL FROM NATURAL GAS USING MILLED CATALYSTS

(71) Applicants: UNIVERSITA' DEGLI STUDI DI UDINE, Udine (IT); BROOKHAVEN SCIENCE ASSOCIATES, LLC, Upton, NY (US)

(72) Inventors: Juan de Jesus JIMENEZ, Middle Island, NY (US); Sanjaya Daminda SENANAYAKE, Great Neck, NY (US); Jose A. RODRIGUEZ, Middle Island, NY (US); Alessandro TROVARELLI, Udine (IT); Sara COLUSSI, Udine (IT); Malia DANIELIS, Udine (IT)

(21) Appl. No.: 19/119,492

(22) PCT Filed: Oct. 9, 2023
(86) PCT No.: PCT/IB2023/060099

§ 371 Date: Apr. 9, 2025

(30) Foreign Application Priority Data
Oct. 10, 2022 (IT) 102022000020796

(51) Int. Cl. C07C 29/48; B01J 23/63; C07C 31/04

(52) U.S. Cl. CPC C07C 29/48; B01J 23/63; C07C 31/04

(57) ABSTRACT

The disclosure relates to a dry milled catalyst, wherein the dry milled catalyst is a product of dry milling 1) a cerium oxide with 2) rhodium, ruthenium, nickel, cobalt, or a combination thereof. This disclosure relates to a method of converting methane to methanol, comprising heating a reaction mixture comprising methane, hydrogen peroxide, and a dry milled catalyst, wherein the dry milled catalyst is a product of dry milling 1) a cerium oxide with 2) palladium, rhodium, ruthenium, nickel, cobalt, or a combination thereof.

### STATEMENT OF GOVERNMENT LICENSE RIGHTS

The present invention was made with Government support under contract number DE-SC0012704 awarded by the U.S. Department of Energy. The United States government has certain rights in this invention.

### BACKGROUND

The conversion of natural gas to liquid fuels is a central challenge to the catalysis industry at large that remains to be addressed. Specifically, improved methods for the conversion of methane (CH₄), the principal component of natural gas, into a liquid fuel, such as methanol (CH₃OH), are needed. An improved method of converting methane to methanol (MtM) would enable the direct value-added upgrade of methane, which is often flared or discarded, and presents a novel avenue for the generation of methanol, a top 10 global commodity chemical.

Prior attempts to address direct methane to methanol conversion have been mostly focused on using either copper-zeolite based catalysts or noble metal based (Au, Pd, AuPd, Rh, Ir) catalysts supported on either reducible supports or zeolites.

The disadvantages of copper-zeolite or general gas phase direct MtM is the need for sequential oxygen-methane-water cycling, which requires both a change in feedgas composition and operating temperature for each cycle, contributing to catalyst instability. Furthermore, copper catalysts have low intrinsic activity due to the difficulty of cleaving the C—H bond in CH₄ to form the active .CH₃ intermediate. Rhodium based catalysts show promise as a potential MtM catalyst, however, rhodium has an affinity for C—C coupling, making it a more suitable catalyst for the generation of ethanol or acetic acid.

A Pd/ceria catalyst prepared by simple dry milling of metallic Pd and ceria (CeO₂) powders is known in the art. In their paper—Applied Catalysis B. Environmental, vol. 282, 24 Sep. 2020, XP 086353916, ISSN: 0926-3373, DOI: 10.1016/J.APCATB.2020.119567—, Danielis et al. showed that such catalyst has an increased catalytic activity for complete methane oxidation with respect to an analogous sample prepared by traditional incipient wetness impregnation. The improved activity was shown by Danielis et al. to be attributed to the formation of a mixed Pd—CeO₂ amorphous layer on the surface and to be strongly dependent on the milling conditions showing that samples prepared under mild conditions were able to reach full conversion at low temperature compared to samples obtained by high energy milling. In particular, Danielis et al. showed that catalysts containing 4% wt Pd displayed outstanding methane oxidation activity.

However, Danielis et al. show that that the milled catalyst is particularly good at converting methane to CO₂, i.e., fully oxidizes it, which not desirable in methane-to-methanol applications.

A method for directly converting methane in liquid phase methanol using a Rh catalyst, while adding a significantly small amount of hydrogen peroxide is known in the art. KR102117933B1 discloses that the oxygen-oxygen bond of hydrogen peroxide is dissociated to generate methanol through the binding of the hydroxyl groups to the dissociated —CH₃ generated by the methane activation on Rh monoatoms. KR102117933B1 discloses that the support for the catalyst is selected from the group consisting of ZrO₂, CeO₂, TiO₂ and SiO₂. In this regard, KR102117933B1 shows a comparison between Rh-ceria and Rh-zirconia catalyst, wherein Rh-zirconia has been shown to have a better reactivity than Rh-ceria. KR102117933B1 discloses that when the noble metal for the support is Pd, Ir, or Pt it is difficult to generate methanol because the methane does not react with the oxidizing agent.

Therefore, KR102117933B1 shows that Pd is not effective to generate methanol from methane. The most suitable catalyst suggested by KR102117933B1 is Rh-zirconia catalyst.

Accordingly, there is still a need to improve the method of converting methane to methanol, such that it is highly effective and less environmentally impacting.

### SUMMARY

This disclosure relates to a method of converting methane to methanol, comprising heating a reaction mixture comprising methane, aqueous hydrogen peroxide (H₂O₂), and a dry milled catalyst, wherein the dry milled catalyst is a product of dry milling 1) a cerium oxide with 2) either palladium, rhodium, ruthenium, nickel, cobalt, or a combination thereof.

The disclosure relates also to a dry milled catalyst, wherein the dry milled catalyst is a product of dry milling 1) a cerium oxide with 2) rhodium, ruthenium, nickel, cobalt, or a combination thereof.

### BRIEF DESCRIPTION OF THE DRAWINGS

FIG. 1A is a graph depicting direct methane to liquid fuel performance for the milled PdCeO₂ catalyst. Reaction conditions were 25 mg catalyst, 15 mL of 0.5 M H₂O₂ solution, 25 bar operating pressure (20% CH₄ and 80% Ar), and 800 rpm mixing.

FIG. 1B is a graph depicting the influence of time on the direct methane to liquid fuel performance for a PdCeO₂ catalyst prepared by a traditional wetness impregnation technique. Reaction conditions were 25 mg catalyst, 15 mL of 0.5 M H₂O₂ solution, 25 bar operating pressure (20% CH₄ and 80% Ar), 800 rpm mixing, and 125 °C.

FIG. 2 is a graph depicting the influence of time on the direct methane to liquid fuel performance for milled PdCeO₂. Conditions: 25 mg catalyst, 15mL of 0.5 M H₂O₂ solution, 25 bar operating pressure (20% CH₄ and 80% Ar), 800 rpm mixing, 125 °C.

FIG. 3 is a graph depicting the influence of both the metal used for the catalyst and the temperature of the reaction on the direct methane to liquid fuel performance. Reaction conditions were 25 mg catalyst, 15 mL of 0.5 M H₂O₂ solution, 25 bar operating pressure (20% CH₄ and 80% Ar), 800 rpm mixing and either 75 °C or 110 °C.

### DETAILED DESCRIPTION

The method described herein utilizes a milled ceria support that promotes higher catalytic activity for various methane containing reactions and shows excellent stability under rigorous reaction conditions. For example, some methods relate to converting methane to methanol. In such a method, a reaction mixture may be heated, wherein the reaction mixture comprises methane, water, hydrogen peroxide, and a dry milled catalyst. In some embodiments, the dry milled catalyst is a product of dry milling 1) a cerium oxide with 2) palladium, rhodium, ruthenium, nickel, cobalt, or a combination thereof. In other words, in some embodiments, the method of converting methane to methanol, may comprise heating a reaction mixture comprising methane, water (e.g., deionized (DI) water), hydrogen peroxide, and a dry milled catalyst, wherein the dry milled catalyst is a product of dry milling 1) a cerium oxide with 2) palladium, rhodium, ruthenium, nickel, cobalt, or a combination thereof. The catalyst has shown excellent stability under rigorous reaction conditions.

Dry milling of the catalyst produces a catalyst having a structure that is different from other catalysts having the same empirical formula made by a traditional incipient wetness impregnation technique. It is believed that a dry milled catalyst may have a structure comprising a CeO₂ core surrounded by a shell comprising CeO₂ and the noble metal.

In the present description and in appended claims, the term “ceria” refers to cerium(IV) oxide of formula CeO₂ as well as to any non-stoichiometric, anion deficient form of ceria having formula CeO₍₂₋ₓ₎.

In some embodiments of the present invention, there is provided a core-shell catalyst system, characterized in that:
the core may comprise a ceria oxide, wherein said core may comprise at least one particle having size of about 2-100 nm, preferably of about 5-100 nm, more preferably of about 5-80 nm; and
the shell may comprise an amorphous phase comprising a metal selected from palladium, rhodium, ruthenium, nickel, cobalt, or a combination thereof and oxide of ceria, the shell having thickness of ≤about 5 nm, preferably of about 0.1-5 nm.

In one embodiment, the core may comprise ceria.

In one embodiment, the core may consist of at least one oxide selected among ceria, a ceria-based mixed oxide and mixtures thereof, preferably the core may consist of ceria.

In one embodiment, the ceria-based mixed oxide may comprise (be doped with) ≤about 40 mol. %, preferably about 1-40 mol. %, more preferably about 10-30 mol. %, of at least one element selected among Zr, In, Sn, La, Pr, Nd, Pm, Sm, Eu, Gd, Tb, Dy, Ho, Er, Tm, Yb, Lu, Sc, Y and mixtures thereof, preferably Zr.

In some further embodiments of the present invention, the oxide comprised in the core of the catalyst system may have low or high surface area, preferably comprised in the range ≤about 300 m²/g, more preferably about 0.5-300 m²/g, still more preferably about 2.0-100.0 m²/g.

In the core-shell catalyst system of the invention, the shell may preferably comprise the same oxide or oxides comprised in the core of the catalyst system.

In one embodiment, the size of the core may be equal to or greater than the thickness of the shell, preferably the size of the core may be greater than the thickness of the shell.

In some specific embodiments of the present invention, the shell may comprise a metal selected from palladium, rhodium, ruthenium, nickel, cobalt, or a combination thereof in an amount of ≤about 5.0 wt. %, preferably of ≤about 2.0, more preferably of ≤about 1.5 wt. %, still more preferably of about 0.1-1.0 wt. %, wherein the amount of metal corresponds to the nominal load of metal.

In a further embodiment, the shell may comprise at least one further metal M selected among Cu, Ru, Rh, Ag, Os, Ir, Pt, Au and mixture thereof, preferably among Cu, Pt and mixtures thereof, more preferably it is Pt.

In one embodiment, the total metal loading of the catalyst system may be ≤about 5.0 wt. %, preferably ≤about 2.0 wt. %, more preferably ≤about 1.5 wt. %, still more preferably about 0.1-1.0 wt. %, wherein the total metal loading is the sum of the weights of the metals loaded onto the catalyst system, expressed as wt. % with respect to the weight of the catalyst system.

TEM analysis performed on a series of samples showed that no metal is present inside the oxide core of the catalyst system, suggesting that all loaded noble metal is found on the particles surface (i.e. in the shell). Thus, the metal content of the shell corresponds to the nominal metal loading.

In one embodiment, the weight ratio metal selected from palladium, rhodium, ruthenium, nickel, cobalt, or a combination thereof: M (metal(s)) may range from about 90:10 to about 10:90, preferably from about 70:30 to about 30:70, more preferably the weight ratio may be about 50:50.

The weight ratio refers to the ratio between the nominal loading of the metal selected from palladium, rhodium, ruthenium, nickel, cobalt, or a combination thereof and the nominal loading of the second metal M.

In one embodiment, the shell may consist of an amorphous phase comprising a metal selected from palladium, rhodium, ruthenium, nickel, cobalt, or a combination thereof and an oxide as described above.

In one embodiment, the shell may consist of an amorphous phase consisting of a metal selected from palladium, rhodium, ruthenium, nickel, cobalt, or a combination thereof and an oxide as described above.

In one embodiment, the metal selected from palladium, rhodium, ruthenium, nickel, cobalt, or a combination thereof comprised in the shell may be selected among metallic metal, an organic compound of the metal, an inorganic compound of the metal and mixtures thereof, said organic and inorganic compound being solid. Preferably the metal selected from palladium, rhodium, ruthenium, nickel, cobalt, or a combination thereof comprised in the shell may be selected among metallic metal, metal(CH₃COO)₂ and mixtures thereof, more preferably may be metallic metal.

When present, the further metal M comprised in the shell may be selected among metallic M, a solid compound of metal M and mixtures thereof, preferably may be metallic M.

According to a preferred embodiment, the shell of the catalyst system of the invention may comprise metallic Pd and metallic Pt.

In a further aspect, the present invention refers to a method for the preparation of the core-shell catalyst system described above comprising a step (i) of dry grinding a mixture comprising at least one selected among metallic metal selected from palladium, rhodium, ruthenium, nickel, cobalt, or a combination thereof, a solid organic salt of such metal and mixtures thereof and at least one oxide selected among ceria, a ceria-based mixed oxide and mixtures thereof. According to one embodiment, for the preparation of the core-shell catalyst system of the invention wherein the shell comprises a further metal M, the method may comprise a further step (ii) of dry grinding the core-shell catalyst system obtained from step (i) with at least one selected among metallic M, a solid compound of metal M and mixtures thereof, preferably metallic M.

In one embodiment, for the preparation of the core-shell catalyst system wherein the shell comprises a further metal M, the mixture of step (i) may comprise at least one selected among metallic M, a solid compound of metal M and mixtures thereof, preferably metallic M.

In one embodiment, the mixture of step (i) may comprise at least one selected among metallic M, a solid compound of metal M and mixtures thereof, preferably metallic M, and the method may comprise a further step (ii) as described above.

In one embodiment, said at least one further metal M may be selected among Cu, Ru, Rh, Ag, Os, Ir, Pt, Au and mixture thereof, preferably among Cu, Pt and mixtures thereof, more preferably Pt.

In one embodiment, the total metal loading of the catalyst system may be ≤about 5.0 wt. %, preferably ≤about 2.0 wt. %, more preferably ≤about 1.5 wt. %, still more preferably about 0.1-1.0 wt. %, wherein the metal loading is the sum of the weights of the metals loaded onto the catalyst system in step (i) and in step (ii), if present, expressed as wt. % with respect to the weight of the catalyst system.

In one embodiment, the weight ratio between the metal selected from palladium, rhodium, ruthenium, nickel, cobalt, or a combination thereof and M (metal(s)) may range from about 90:10 to about 10:90, preferably from about 70:30 to about 30:70, more preferably the weight ratio may be about 50:50.

The weight ratio refers to the ratio between the nominal loading of the metal selected from palladium, rhodium, ruthenium, nickel, cobalt, or a combination thereof and the nominal loading of the second metal M.

In one embodiment, the amount of metal selected from palladium, rhodium, ruthenium, nickel, cobalt, or a combination thereof in the mixture of the dry grinding step (i) may be ≤about 5.0 wt. %, preferably ≤about 2.0 wt. %, more preferably ≤about 1.5 wt. %, still more preferably about 0.1-1.0 wt. %.

The metal compound of step (i) may be selected among metallic metal, metal(CH₃COO)₂ and mixtures thereof, preferably may be metallic metal. When present, the solid compound of the further metal M may be selected among an organic compound of M, an inorganic compound of M and mixtures thereof.

In a preferred embodiment, the dry-grinding step (i) and/or (ii) may be carried out at temperature of about 20-35 °C and pressure of about 101.3 kPa.

In one embodiment, the dry grinding step (i) and the dry grinding step (ii), if present, may be carried out by ball-milling.

In one embodiment, the dry grinding step (i) and the dry grinding step (ii), if present, may be carried out using only one grinding ball.

In one embodiment, the weight ratio of grinding media to powders may be comprised in the range of about 5-20, preferably may be about 10. A higher ball-to-powder weight ratio induces a noticeable worsening of the catalytic activity.

In one embodiment, the dry grinding step (i) and the dry grinding step (ii), if present, may be carried out for a time comprised of about 5-20 minutes. Surprisingly, it has been found that milling times lower than 5 minutes gave unsatisfactory results as the contact between metal and support was insufficient to obtain an optimized shell layer, whereas longer milling time induces a noticeable worsening of the catalytic activity.

According to a preferred embodiment, the method for the preparation of the core-shell catalyst system may comprise a step (a) of calcining the at least one oxide selected among ceria, a ceria-based mixed oxide and mixtures thereof, wherein step (a) is carried out before the dry grinding step (i). The calcining step (a) may be carried out in presence of oxygen, more preferably of air. In a further preferred embodiment, the calcining step (a) may be carried out at temperature ≥about 350 °C, preferably of about 350 °-1200 °C, more preferably of about 500 °-1000 °C, still more preferably of about 900 °C, for a time ≥about 2 hours, preferably of about 2-4 hours, more preferably of about 3 hours.

In one embodiment, the at least one ceria-based mixed oxide used in the dry grinding step (i) may comprise (be doped with) ≤about 40 mol. %, preferably about 1-40 mol. %, more preferably about 10-30 mol. %, of at least one element selected among Zr, In, Sn, La, Pr, Nd, Pm, Sm, Eu, Gd, Tb, Dy, Ho, Er, Tm, Yb, Lu, Sc, Y and mixtures thereof, preferably Zr.

In one embodiment, the oxide of the dry grinding step (i) may have low or high surface area, preferably comprised in the range ≤about 50 m²/g, more preferably about 0.5-50 m²/g, still more preferably about 2.0-30.0 m²/g.

In one embodiment, the dry grinding step (i) and the dry grinding step (ii), if present, may be carried out under low energy milling. According to a preferred embodiment, the dry grinding steps (i) and (ii), if present, may be carried out in a ball-mill oscillating at a frequency of about 10-30 Hz, preferably of about 15 Hz.

Optionally, the raw materials used in the method of the invention may be pre-mixed without grinding to homogenize the powders mixture.

In a further aspect, the present invention refers to a core-shell catalyst system obtained/obtainable from the method as described above in which M is Rh, Ru, Ni, Co, or a combination thereof.

Advantageously, with the method of the present invention a catalyst is formed, that has unique features and a higher catalytic activity compared to traditional metal-ceria catalysts prepared by conventional wet methods. The core-shell catalyst system of the invention is obtained/obtainable by a fast, low energy milling method without the need of solvents or any thermal treatment.

The core-shell catalytic system of the disclosure in which M is Pd, Rh, Ru, Ni, Co, or a combination thereof has been proven to be highly active in a method to convert methane to methanol.

In some embodiments, the catalyst has an empirical formula of MxCeO₂. With respect to the empirical formula MₓCeO₂, M is Pd, Rh, Ru, Ni, Co, or a combination thereof. In some embodiments, M is rhodium, nickel, or a bimetallic combination such as AuPd or PdZn.

With respect to the empirical formula MₓCeO₂, in some embodiments, M is Pd.

With respect to the empirical formula MₓCeO₂, in some embodiments, M is Rh.

With respect to the empirical formula MₓCeO₂, in some embodiments, M is Ru.

With respect to the empirical formula MₓCeO₂, in some embodiments, M is Ni.

With respect to the empirical formula MₓCeO₂, in some embodiments, M is Co.

With respect to the empirical formula MₓCeO₂, in some embodiments, M is a bimetallic combination.

With respect to the empirical formula MₓCeO₂, in some embodiments, M is AuPd.

With respect to the empirical formula MₓCeO₂, in some embodiments, M is PdZn.

With respect to the empirical formula MₓCeO₂, the value of x may be about 0.001-0.1, such as about 0.001-0.005, about 0.005-0.02, about 0.02-0.05, or about 0.05-0.1.

With respect to the empirical formula MₓCeO₂, in some embodiments, M is Pd and x is about 0.001-0.1.

With respect to the empirical formula MₓCeO₂, in some embodiments, M is Pd and x is about 0.001-0.005.

With respect to the empirical formula MₓCeO₂, in some embodiments, M is Pd and x is about 0.005-0.02.

With respect to the empirical formula MₓCeO₂, in some embodiments, M is Pd and x is about 0.02-0.05.

With respect to the empirical formula MₓCeO₂, in some embodiments, M is Pd and x is about 0.05-0.1.

The milled support material is generally loaded with some weight percent of a noble metal, such that the empirical formula of the product after milling is MₓCeO₂ as described in the paragraphs above.

Any suitable amount of catalyst may be used in the reaction mixture, such as about 0.01-10%, about 0.05-0.5% (e.g., 25 mg of catalyst in 15 mL of liquid), about 0.5-2%, about 2-5%, or about 5-10%, based upon the weight of the liquid (e.g., aqueous hydrogen peroxide) in the reaction mixture.

Upon mixing the catalyst in a solution of hydrogen peroxide and pressurized methane, the catalyst and hydrogen peroxide convert the methane into methanol, formic acid (HCOOH), carbon dioxide (CO₂), and residual oxygen (O₂).

For some reactions, the methane is in the form of a pressurized gas, such as in a pressurized gas mixture. The pressurized gas or pressurized gas mixture may have any suitable pressure, such as about 1-100 bar, about 1-10 bar, about 10-40 bar, about 40-70 bar, or about 70-100 bar.

In the pressurized gas mixture, any suitable amount of or concentration methane may be used, such as about 3-80%, about 3-10%, about 10-30%, about 30-50%, or about 50-80% methane, based upon the molar percentage of methane in the pressurized gas. The remaining gas may be, e.g., an inert gas such as N₂, He, Ne, Ar, etc. In some embodiments, the methane is mixed with Ar gas, e.g., at about 10-30% molar percentage.

In some embodiments, the reaction mixture containing the catalyst, hydrogen peroxide and pressurized methane further comprises water, such as deionized water, so that the reaction mixture is aqueous.

Any suitable amount or concentration of hydrogen peroxide may be used, such as about 0.2-1 M, about 0.2-0.4 M, about 0.4-0.6 M, about 0.6-0.8 M, or about 0.8-1 M.

The reaction mixture may be heated or maintained at a temperature of, e.g., about 30-200 °C, about 25-150 °C, about 25-75 °C, about 50-100 °C, about 100-150 °C, about 100-120 °C, about 120-130 °C, or about 150-200 °C.

Mixing may occur during the heating, e.g., at about 100-2000 rpm, about 100-500 rpm, about 500-1000 rpm, or about 1000-2000 rpm.

The reaction mixture may be heated or maintained at an elevated temperature described above for any suitable amount of time, such as about 5 hours or less, about 0.5-3 hours, about 0.1-1 hours, 1-2 hours, 1-3 hours, or 3-5 hours.

In some embodiments, the catalyst is PdCeO₂ in an amount that is about 0.5-2% of the weight (e.g., about 1.7% by weight) of a 0.2-0.4 M aqueous hydrogen peroxide solution (e.g., about 0.5 M), the reaction mixture has a pressure of 10-40 bar (e.g., 25 bar) under a mixed gas of 10-30% methane (e.g., 20% methane at a partial pressure of 5 bar) in argon (e.g. 80% argon at a partial pressure of 20 bar), and the reaction is maintained at a temperature of about 100-120 °C (e.g., 110 °C) for about 1-3 hours.

The present invention is further described by not limited to the following examples.

### Example 1

The catalyst is dispersed in a 0.5 M H₂O₂ aqueous solution then sealed in an autoclave reactor and pressurized to the desired pressure under a range of methane gas concentrations (3%-80% methane). The reactor is brought up to temperature using a block heater as part of, e.g., a commercial Parr Reactor Batch Reactor set up. Once the reaction is at temperature (Temp=25-150 °C) the mixing is turned on at a rate of 800 rpm. The reaction is then allowed to process for a set amount of time (time=30-180 minutes) under a fixed temperature and mixing speed. Gaseous products are analyzed online before and after reaction with a GC and a MS for gaseous product quantification and identifying, respectively. Liquid products are quantified via ¹H-NMR using a known concentration of sodium trimethylsilylpropanesulfonate mixed with deuterium oxide as an internal standard. All gas and liquid phase sampling after reaction are carried out at <10 °C to mitigate methanol volatilization, where the reactor is chilled with an ice bath upon the completion of the reaction.

### Example 2

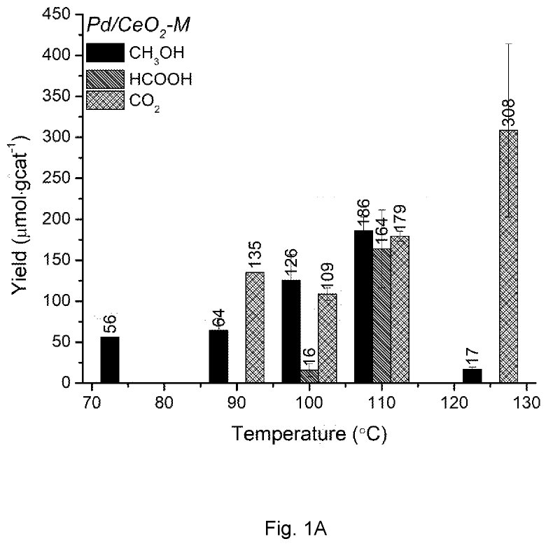

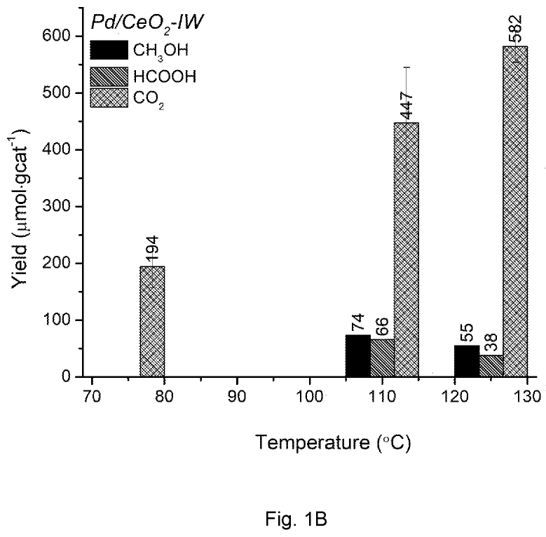

Using the method described in Example 1, the milled MCeO₂ catalysts (Pd/CeO₂-M, FIG. 1A) were tested against a reference catalyst with the same metal concentration prepared via a traditional incipient wetness (Pd/CeO₂-IW, FIG. 1B). Reaction conditions were 25 mg catalyst, 15 mL of 0.5 M H₂O₂ solution, 25 bar operating pressure (20% CH₄ and 80% Ar), 800 rpm mixing, and 125 °C.

The milled catalyst yielded a factor of two improved total oxygenate yield (350 μmol·g_{cat}⁻¹ vs 142 μmol·g_{cat}⁻¹) as compared to the catalyst prepared by the wet impregnation technique.

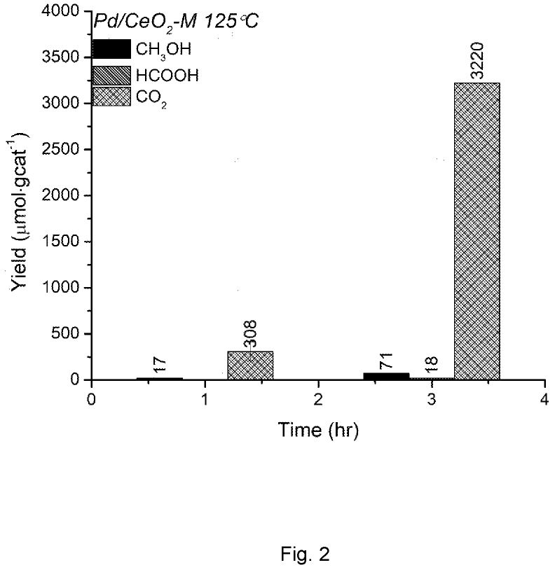

The total oxygenate yield for impregnated Pd/CeO₂ at 110 °C was (142 μmol·g_{cat}⁻¹ total, 74.8 μmol_{MeOH}·g_{cat}⁻¹ & 68.4 μmol_{FA}·g_{cat}⁻¹). Furthermore, the suppression of CO₂ formation, an undesired product, is favored with the milled catalysts at all observed temperatures relative to the impregnated catalyst. The difference between the catalytic properties of the dry milled catalyst as compared to the reference catalyst demonstrates that the method of preparing the catalyst affects the structure of the catalyst. At 110 °C, not only is the oxygenate yield of the milled Pd/CeO₂ nominally a factor of two higher than the control impregnated catalysts but the formation of CO₂ is considerably diminished (179 μmol·g_{cat}⁻¹ vs 447 μmol·g_{cat}⁻¹ for the milled and impregnated; respectively). Additionally, the influence of time on the product distribution was also observed for the milled Pd/CeO₂ catalysts at 125 °C, shown in FIG. 2, where increasing the time from one hour to three hours of reaction time results in an order of magnitude increase in CO₂ formation, where oxygenate yield only increased by approximately a factor of four. This shows that the CO₂ is the terminal product in the direct methane to methanol conversion, where the successive oxidation of methanol, formic acid, or simply the overoxidation of methyl species results in the formation of CO₂.

### Example 3

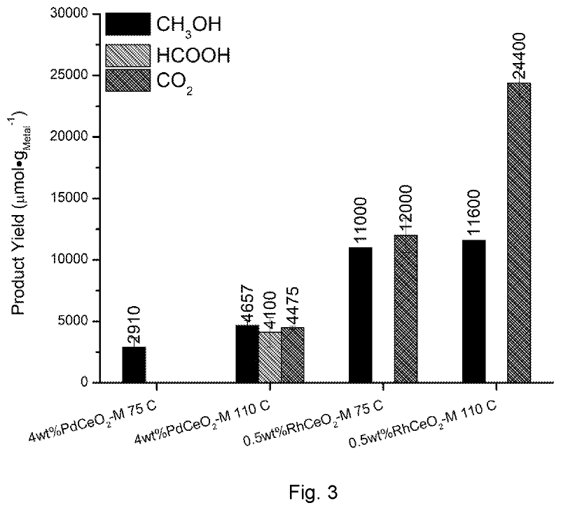

Using the method described in Example 1, a comparison between a MCeO₂ catalyst based on M=Pd (Pd/CeO₂-M, FIG. 3) and a MCeO₂ catalyst based on M=Rh (Rh/CeO₂-M, FIG. 3) was made. The weight percentages of metal used for the Pd-catalyst and the Rh-catalyst were 4% by weight (wt) and 0.5% by weight (wt) respectively. The activity based on weight of metal was then normalized in order to allow the comparison. Reaction conditions were 25 mg catalyst, 15 mL of 0.5 M H₂O₂ solution, 25 bar operating pressure (20% CH₄ and 80% Ar), 800 rpm mixing, and either 75 °C or 110 °C.

Depending on the metal used for the catalyst, the methanol yield differs, as well as the amounts of side-products CO₂ and formic acid. The use of Rh leads to higher methanol yields regardless of the temperature of the reaction, but at the same time, higher amounts of CO₂ are produced.

Furthermore, the data showed that the yields are also dependent on the temperature. At 75 °C, when the Pd catalyst is used, neither CO₂ nor formic acid are produced, while when the Rh catalyst is used, no formic acid is produced but CO₂ is produced in an amount higher than methanol. At 110 °C, when the Pd catalyst is used methanol, formic acid, and CO₂ are produced in a comparable amount. While, if the Rh catalyst is used, no formic acid is produced but CO₂ forms in an amount that is twice as higher as methanol.

1-4. (canceled)

5. A method of converting methane to methanol, comprising heating a reaction mixture comprising methane, water, hydrogen peroxide, and a dry milled catalyst, wherein the dry milled catalyst is a product of dry milling 1) a cerium oxide with 2) palladium, rhodium, ruthenium, nickel, cobalt, or a combination thereof.

6. The method of claim 5, wherein the dry milled catalyst is represented by a formula MₓCeO₂, wherein M is Pd, Rh, Ru, Ni, Co, or a combination thereof, and x is about 0.001 to about 0.1.

7. The method of claim 6, wherein M is Pd.

8. The method of claim 5, wherein the reaction mixture is aqueous.

9. The method of claim 5, wherein the methane is in the form of a pressurized gas.

10. The method of claim 9, wherein the methane is in a pressurized gas mixture containing about 3% to about 80% molar percentage methane.

11. The method of claim 9, wherein the pressurized gas has a pressure of about 10 bar to about 40 bar.

12. The method of claim 5, wherein the dry milled catalyst is about 0.05% to about 0.5% of the weight of all liquid in the reaction mixture.

13. The method of claim 5, wherein the hydrogen peroxide in the reaction mixture has a concentration of about 0.2 M to about 1 M.

14. The method of claim 5, wherein the reaction mixture is heated at a temperature of about 150 °C or less.

15. The method of claim 14, wherein the reaction mixture is heated at a temperature of about 100 °C to about 120 °C.

16. The method of claim 5, wherein the reaction mixture is heated for a period of about 5 hours or less.

17. The method of claim 5, wherein the reaction mixture is heated for a period of about 1 hour to about 3 hours.
